SEQUENCE LISTING (13 sequences)

SEQ ID NO: 1 - protein, 269 aa, synthetic construct
ETADATRASVAELARVSDAVLDADVPGTAWYTDAESGKLVVTADATVSAAELAQLKKAAGDKAGAVEIKRTPGTFNKLIAGGEAIYAAGGGRSSLGFNVRSSSGATYALTAGHCTEIASTWYTNSGQTSLAGTRAGTSFPGNDYGLIRHSNASAADGRVYLYNGSYRDITGAGNAYVGQTVQRSGSTTGLHSGRVTGLNATVNYGGGDIVSGLIQTNVCAEPGDSGGALFAGSTALGLTSGGSGNCRTGGTTFFQPVTEALSAYGVSII

SEQ ID NO: 2 - protein, 269 aa, synthetic construct
ETADATRASVAELARVSDAVLDADVPGTAWYTDAESGKLVVTADATVSAAELAQLKKAAGDKAGAVEIKRTPGTFNKLIAGGEAIYAAGGGRSSLGFNARSSSGATYALTAGHCTNGASTWYTNSGQTSLAGTRAGTSFPGNDYGLIRHSNASAADGRVSLYNGSYRDITGAGNAYVGQTVQRSGSTTGLHSGRVTGLNATVNYGGGDIVSGLIQTNVCAEPGDSGGALFAGSTALGLTSGGSGNCRTGGTTFFQPVTEALSAYGVSII

SEQ ID NO: 3 - DNA, 810 bp, synthetic construct
  source 1..810: mol_type = other DNA
gagaccgccgacgccacccgcgcctccgtcgccgagctggcccgcgtctccgacgccgtcctggacgccgacgtcccgggcaccgcctggtacaccgacgccgagtccggcaagctggtcgtcaccgccgacgccaccgtctccgccgccgagctggcccagctgaagaaggccgccggcgacaaggccggcgccgtcgagatcaagcgcaccccgggcaccttcaacaagctgatcgccggcggcgaggccatctacgccgccggcggcggccgctcctccctgggcttcaacgcgcgctcctcctccggcgccacctacgccctgaccgccggccactgcaccaacggcgcctccacctggtacaccaactccggccagacctccctggccggcacccgcgccggcacctccttcccgggcaacgactacggcctgatccgccactccaacgcctccgccgccgacggccgcgtctcgctgtacaacggctcctaccgcgacatcaccggcgccggcaacgcctacgtcggccagaccgtccagcgctccggctccaccaccggcctgcactccggccgcgtcaccggcctgaacgccaccgtcaactacggcggcggcgacatcgtctccggcctgatccagaccaacgtctgcgccgagccgggcgactccggcggcgccctgttcgccggctccaccgccctgggcctgacctccggcggctccggcaactgccgcaccggcggcaccaccttcttccagccggtcaccgaggccctgtccgcctacggcgtctccatcatctga

SEQ ID NO: 4 - DNA, 84 bp, synthetic construct
  source 1..84: mol_type = other DNA
atgcgtcgcaccctcaaggccgtgggagcagccgcggcggcggccacctgcgtcctcgccgcgacggcaggcaccgcgcaggcc

SEQ ID NO: 5 - DNA, 97 bp, synthetic construct
  source 1..97: mol_type = other DNA
tgttcacattcgaacggtctctgctttgacaacatgctgtgcggtgttgtaaagtcgtggccaggagaatacgacagcgtgcaggactgggggagtt

SEQ ID NO: 6 - DNA, 42 bp, synthetic construct
  source 1..42: mol_type = other DNA
agcagtgctgaaggccgtcaaggggcgttcgtggtgggaaga

SEQ ID NO: 7 - DNA, 1033 bp, synthetic construct
  source 1..1033: mol_type = other DNA
tgttcacattcgaacggtctctgctttgacaacatgctgtgcggtgttgtaaagtcgtggccaggagaatacgacagcgtgcaggactgggggagttatgcgtcgcaccctcaaggccgtgggagcagccgcggcggcggccacctgcgtcctcgccgcgacggcaggcaccgcgcaggccgagaccgccgacgccacccgcgcctccgtcgccgagctggcccgcgtctccgacgccgtcctggacgccgacgtcccgggcaccgcctggtacaccgacgccgagtccggcaagctggtcgtcaccgccgacgccaccgtctccgccgccgagctggcccagctgaagaaggccgccggcgacaaggccggcgccgtcgagatcaagcgcaccccgggcaccttcaacaagctgatcgccggcggcgaggccatctacgccgccggcggcggccgctcctccctgggcttcaacgcgcgctcctcctccggcgccacctacgccctgaccgccggccactgcaccaacggcgcctccacctggtacaccaactccggccagacctccctggccggcacccgcgccggcacctccttcccgggcaacgactacggcctgatccgccactccaacgcctccgccgccgacggccgcgtctcgctgtacaacggctcctaccgcgacatcaccggcgccggcaacgcctacgtcggccagaccgtccagcgctccggctccaccaccggcctgcactccggccgcgtcaccggcctgaacgccaccgtcaactacggcggcggcgacatcgtctccggcctgatccagaccaacgtctgcgccgagccgggcgactccggcggcgccctgttcgccggctccaccgccctgggcctgacctccggcggctccggcaactgccgcaccggcggcaccaccttcttccagccggtcaccgaggccctgtccgcctacggcgtctccatcatctgaagcagtgctgaaggccgtcaaggggcgttcgtggtgggaaga

SEQ ID NO: 8 - DNA, 33 bp, synthetic construct
  source 1..33: mol_type = other DNA
ccatcatctgaagcagtgctgaaggccgtcaag

SEQ ID NO: 9 - DNA, 34 bp, synthetic construct
  source 1..34: mol_type = other DNA
gtgcgacgcataactcccccagtcctgcacgctg

SEQ ID NO: 10 - DNA, 31 bp, synthetic construct
  source 1..31: mol_type = other DNA
gggggagttatgcgtcgcaccctcaaggccg

SEQ ID NO: 11 - DNA, 32 bp, synthetic construct
  source 1..32: mol_type = other DNA
cagcactgcttcagatgatggagacgccgtag

SEQ ID NO: 12 - DNA, 22 bp, synthetic construct
  source 1..22: mol_type = other DNA
ctgggggagttatgcgtcgcac

SEQ ID NO: 13 - DNA, 21 bp, synthetic construct
  source 1..21: mol_type = other DNA
cagcactgcttcagatgatgg

(19) United States
(12) Patent Application Publication
(10) Pub. No.: US 2026/0347822 A1
(43) Pub. Date: Nov. 5, 2026

(54) KERATINASE MUTANT, ITS ENCODING GENE, AN EXPRESSION VECTOR, AND USES THEREOF

(71) Applicant: Hubei Kaitai Biotechnology Co., Ltd., Shiyan (CN)

(72) Inventors: KAI BAO, Shiyan (CN); CHUNQIANG HE, Shiyan (CN); ZIQI WU, Shiyan (CN); LILI BAO, Shiyan (CN); RUOKUN WU, Shiyan (CN); ZEHUI WU, Shiyan (CN); XIN FANG, Shiyan (CN)

(73) Assignee: Hubei Kaitai Biotechnology Co., Ltd., Shiyan (CN)

(21) Appl. No.: 19/537,659

(22) Filed: Feb. 12, 2026

(30) Foreign Application Priority Data
Apr. 24, 2025 (CN) 2025105187027

(51) Int. Cl. C12N 9/64; C12N 1/20; C12N 15/76; C12R 1/465

(52) U.S. Cl. CPC C12N 9/6424; C12N 1/20; C12N 15/76; C07K 2319/02; C12R 2001/465; C12Y 304/21

(57) ABSTRACT

The present disclosure pertains to the technical field of genetic engineering, and more particularly, to a keratinase mutant, its encoding gene, an expression vector, and uses thereof. The amino acid sequence of the keratinase mutant is set forth in SEQ ID NO: 2. The keratinase mutant can effectively degrade keratin substrates, exhibits high catalytic degradation activity toward keratin, and shows a significant increase in enzymatic activity. Moreover, the keratinase mutant is adapted to use keratin-containing wastes as direct substrates; when used in combination with a reducing agent, it can decompose an entire feather within 24 hours. This markedly improves the degradation efficiency of keratin-containing wastes, significantly lowers both the production cost of keratinase and the processing cost of keratin-containing waste, and thereby demonstrates broad market prospects and notable industrial applicability in facilitating the degradation of feather wastes, as well as the conversion, recovery, and reuse of keratin resources.

### CROSS-REFERENCE TO RELATED APPLICATIONS

The application claims priority to Chinese patent application No. 202510518702.7, filed on Apr. 24, 2025, with the entire contents incorporated herein by reference.

### SEQUENCE LISTING

The sequence listing xml file submitted herewith, named “TYUS2520_Sequence_Listing.xml”, created on Jan. 30, 2026, and having a file size of 15,019 bytes, is incorporated by reference herein.

### TECHNICAL FIELD

The present disclosure relates to the technical field of genetic engineering, and in particular to a keratinase mutant, its encoding gene, an expression vector, and uses thereof.

### BACKGROUND

Keratin is an insoluble protein that is widely present in hard structures of animals, such as feathers, hair, wool, nails, horns, hooves, and scales. The crude protein content of such structures exceeds 80%, the total amino acid content is over 70%, and the content of essential amino acids is extremely high, making keratin a potential high-quality natural protein source. However, the stable chemical structure of keratin renders it recalcitrant to degradation, which hinders the resource utilization of these keratin-containing wastes. For example, feathers are by-products of poultry processing rich in keratin. Except for some waterfowl feathers used in high-value-added products such as down products and decorations, a large number of feathers are not reasonably utilized and are instead directly discarded, landfilled, or incinerated as garbage, resulting in ecological environment pollution and serious waste of protein resources.

Current research has explored the degradation or partial degradation of waste feathers into a mixture containing soluble keratin, amino acids, and peptides, which is used as a feed protein source and soil amendment to promote plant and animal growth. Additionally, feather keratin is also rich in umami amino acids such as glutamic acid and aspartic acid, making it suitable for producing novel food additives in the food processing industry. Amino acids and their derivatives obtained from keratin hydrolysis can also be applied in medicine, textiles, leather processing, composite materials industries, among others. Therefore, research on the conversion of keratin resources to achieve high-value recycling of waste resources holds significant practical importance and is attracting increasing attention.

Traditional methods for decomposing feathers mainly rely on physicochemical methods, such as high temperature and high pressure, acid-alkali treatment, etc. However, these methods suffer from drawbacks such as high energy consumption and substantial loss of nutrients. In contrast, enzymatic degradation of keratin offers a more promising solution due to its low cost, mild treatment conditions, effective preservation of protein and amino acid resources in feathers, and environmentally friendly production process. Keratinase is a protease capable of specifically degrading keratin. It can be produced by various microorganisms including bacteria, fungi, and actinomycetes, and exhibits broad substrate specificity. Under specific pH and temperature conditions, keratinase can efficiently hydrolyze keratin and related proteins from sources such as feathers, wool, keratinized tissues, human hair, and nails, yielding high-value bioactive peptides and free amino acids without disrupting the natural structure of the amino acids. Thus, it is considered an ideal catalyst for the conversion of keratin-containing wastes.

Keratinase-producing bacteria are widely sourced and distributed. However, wild-type keratinase-producing strains have low enzyme yield, and the keratinases they produce often suffer from insufficient catalytic efficiency and require prolonged hydrolysis times for keratin degradation, which significantly restricts the development and application of keratinases. Therefore, improving the activity of keratinase to facilitate the high-value utilization of keratin-containing wastes, such as feathers, has good market application prospects and profound socio-economic benefits.

### SUMMARY

In view of the above technical problems in the prior art, such as insufficient keratinase activity, the present disclosure provides a novel keratinase mutant, as well as a nucleic acid molecule containing a gene encoding said mutant, an expression vector comprising said nucleic acid molecule, and a recombinant strain. Furthermore, the present disclosure provides the use of the keratinase mutant in treating keratin-containing substances.

To achieve the above objective, the present disclosure is specifically achieved through the following schemes:

In a first aspect, the present disclosure provides a keratinase mutant, the amino acid sequence of which is set forth in SEQ ID NO: 2.

In a second aspect, the present disclosure provides a nucleic acid molecule containing a gene sequence encoding the keratinase mutant as mentioned above.

Further, the gene sequence encoding the keratinase mutant is set forth in SEQ ID NO: 3.

Further, the nucleic acid molecule further contains gene sequences encoding a signal peptide, a promoter, and/or a terminator; the keratinase mutant is located downstream of the signal peptide, the promoter is located upstream of the signal peptide, and the terminator is located downstream of the keratinase mutant.

Furthermore, the signal peptide is a vsi signal peptide with the gene sequence set forth in SEQ ID NO: 4; the promoter is a kasOp* promoter with the gene sequence set forth in SEQ ID NO: 5; and the terminator is a T1 terminator with the gene sequence set forth in SEQ ID NO: 6.

In a third aspect, the present disclosure provides an expression vector comprising the nucleic acid molecule as mentioned above.

Further, the expression vector is a Streptomyces integrative vector pSET152.

In a fourth aspect, the present disclosure provides a recombinant strain carrying the expression vector as mentioned above.

Further, the recombinant strain is Streptomyces lividans TK24.

In a fifth aspect, the present disclosure provides the use of the keratinase mutant as mentioned above in the treatment of keratin-containing substances.

Further, the keratin-containing substances are selected from the group consisting of feathers, hair, or wool; and more particularly, selected from avian feathers.

Further, a method for treating the keratin-containing substances with the keratinase mutant comprising:
(a) mixing the keratinase mutant or a crude enzyme solution thereof with the keratin-containing substances;
(b) adding a reducing agent;
and (c) performing a catalytic reaction at a temperature ranging from 50 °C to 60 °C.

Furthermore, the crude enzyme solution of the keratinase mutant is obtained by a process comprising:
(i) culturing the recombinant strain according to the fourth aspect to produce a fermentation broth;
(ii) centrifuging the fermentation broth to obtain a supernatant;
(iii) filtering the supernatant;
and (iv) collecting the filtrate to obtain the crude enzyme solution containing the keratinase mutant.

Furthermore, the reducing agent is dithiothreitol (DTT).

The advantages and positive effects of the present disclosure are as follows:

The keratinase mutant of the present disclosure can effectively degrade keratin substrates, exhibits high catalytic degradation activity toward keratin, and shows a significant increase in enzymatic activity compared to that of the wild-type keratinase. Moreover, the keratinase mutant is adapted to use keratin-containing wastes (including but not limited to feathers) as direct substrates; when used in combination with the reducing agent dithiothreitol (DTT), it can substantially decompose an entire feather within 24 hours. This markedly improves the degradation efficiency of keratin-containing wastes such as feathers, significantly lowers both the production cost of keratinase and the processing cost of keratin-containing waste, and thereby demonstrates broad market prospects and notable industrial applicability in facilitating the degradation of feather wastes, as well as the conversion, recovery, and reuse of keratin resources.

### BRIEF DESCRIPTION OF DRAWINGS

To more clearly illustrate the technical solutions in the embodiments of the present disclosure, the accompanying drawings used in the embodiments are briefly described below. Apparently, the accompanying drawings in the following description are merely some embodiments of the present disclosure, and for those of ordinary skill in the art, other accompanying drawings can also be obtained based on these drawings without exerting creative efforts.

FIG. 1 is a map of the expression vector for the wild-type keratinase KT-ker;

FIG. 2 is a map of the expression vector for the keratinase mutant KT1113;

FIG. 3 is a polyacrylamide gel electrophoresis (SDS-PAGE) image of the crude enzyme solution of the keratinase mutant KT1113;

FIG. 4 is an image showing the enzymatic hydrolysis effect of the crude enzyme solution of the keratinase mutant KT1113 on feathers within 24 hours.

### DETAILED DESCRIPTION OF THE EMBODIMENTS

To clarify the objectives, technical solutions, and advantages of the present disclosure, the present disclosure is further described below in conjunction with the embodiments. Unless otherwise specified, all equipment and reagents used in the embodiments and experimental examples are commercially available. The embodiments described herein are intended to explain the present disclosure and not to limit it.

Based on the information contained in the present disclosure, those skilled in the art may easily make various modifications to the precise description of the present disclosure without departing from the spirit and scope of the appended claims. It should be understood that the scope of the present disclosure is not limited to the defined processes, properties, or components, as these embodiments and other descriptions are provided merely to illustrate specific aspects of the present disclosure. In fact, any modifications obviously made to the embodiments of the present disclosure by those skilled in the art or related fields all fall within the scope of the appended claims.

To better understand the present disclosure rather than limit its scope, all numbers indicating quantities, percentages, and other numerical values used in the present disclosure shall be understood as being modified by the term “about” in all cases. Therefore, unless otherwise specified, the numerical parameters listed in the specification and the appended claims are approximations, which may be changed depending on the desired ideal properties. Each numerical parameter should at least be considered as being obtained based on reported significant figures and conventional rounding methods.

In addition, it should be noted that, unless otherwise defined, the scientific and technical terms used in the context of the present disclosure shall have the meanings commonly understood by those of ordinary skill in the art.

The terms “including”, “comprising”, “containing”, “having” and other similar expressions have non-restrictive meanings, i.e., other steps and components that do not affect the results may be added.

The term “and/or” should be regarded as the specific disclosure of each of the designated features or components either with or without the other. For example, “A and/or B” will be regarded as including the following situations: (i) A alone, (ii) B alone, and (iii) both A and B together.

The term “gene” refers to the complete nucleotide sequence required for producing a polypeptide chain or a functional RNA. A gene may comprise several operably linked nucleic acid fragments, such as a 5′ non-coding region, a coding sequence, and a 3′ non-coding region containing a polyadenylation site. Untranslated regions typically possess regulatory functions for gene expression, such as promoters and terminators.

The term “fusion gene” refers to any gene that is not naturally occurring, comprising regulatory sequences and coding sequences that do not exist together in nature. Thus, a fusion gene may include regulatory sequences and coding sequences derived from different organisms, or regulatory sequences and coding sequences derived from the same organism but arranged in a manner different from that in nature, such as the keratinase mutant expression cassette of the present disclosure.

The term “promoter” refers to a DNA sequence that controls the transcription of one or more nucleic acids or genes, usually located upstream of the transcription start site of a gene in the direction of transcription (i.e., at the 5′ end of the coding sequence). The promoter itself is not transcribed and is structurally identified by the presence of a binding site for DNA-dependent RNA polymerase and a transcription start site, and may also contain any other DNA sequences, including but not limited to transcription factor binding sites, repressor and activator protein binding sites, and any other nucleotide sequences known to those of ordinary skill in the art that directly or indirectly act to regulate the amount of transcription from the promoter.

The term “terminator” refers to a DNA sequence that provides a termination signal during transcription, located at the 3′ end of the coding sequence. At the RNA level, it functions by forming a stem-loop structure from the transcribed terminator sequence to terminate RNA synthesis.

The term “vector” refers to a self-replicating DNA molecule used to transfer a target gene into a host cell, and is often in the form of a circular double-stranded DNA molecule. A vector containing an exogenous gene is referred to as a recombinant vector.

The term “expression vector” allows the target gene inserted into the vector to be expressed in a host cell, and comprises regulatory elements for expression in a specified host cell, such as a promoter and/or a terminator. The expression vector is introduced into an appropriate host cell to enable the expression of the inserted target gene.

The terms “introduction” or “transfer” refer to the transfer of a target gene into a host cell, resulting in the stable inheritance of the gene. The introduced nucleic acid molecule may be retained in the host cell in the form of a plasmid, or may be integrated into the host cell genome. Nucleic acid molecules and/or vectors can be introduced into host cells by methods such as “transfection”, “transformation”, or “transduction”. A host cell containing the introduced nucleic acid molecule is referred to as a “transgenic”, “recombinant”, or “transformed” organism or an “engineered” organism. The introduction of vectors into host cells can be performed using conventional techniques well known to those of ordinary skill in the art.

Unless otherwise specified, similar terms such as “nucleotide”, “nucleic acid”, “nucleic acid molecule”, and “nucleic acid fragment” may be used interchangeably in the context of the present disclosure. The terms “gene,” “nucleic acid sequence,” “nucleotide sequence,” or “nucleic acid molecule” as used herein refer to a polymer of nucleotides of any length, which may be ribonucleotides or deoxyribonucleotides, and the terms refer only to the primary structure of the molecule.

To make the above objectives, features and advantages of the present disclosure easier to understand, the present disclosure is described in detail below.

Keratinase is a protease capable of specifically degrading keratin, which is primarily produced by microorganisms including fungi, actinomycetes, and bacteria. For example, it can degrade keratin from sources such as feathers, wool, keratinized tissues, human hair, and nails into bioactive peptides and free amino acids. Therefore, keratinase exhibits broad application prospects in various industries, such as feed additives, soil amendments, food additives, leather softening, cosmetics, and pharmaceuticals, as well as in environmental remediation.

In comparison to conventional physicochemical methods for decomposing keratin, enzymatic degradation using keratinase offers significant advantages, including mild treatment conditions, effective preservation of protein, peptide, and amino acid resources in keratin-containing wastes such as feathers, and a more environmentally friendly production process. These attributes are critical for achieving the high-efficiency and high-value utilization of keratin resources. Therefore, keratinase is regarded as an ideal catalyst for the conversion of keratin-containing wastes and has garnered considerable attention in recent years. Nonetheless, currently reported keratinases possess insufficient catalytic efficiency and require prolonged hydrolysis times for keratin substrates such as feathers, which renders them unsuitable for large-scale industrial applications. Therefore, the systematic screening, investigation, and development of novel keratinases to enhance their catalytic activity, and the utilization of high-efficiency keratinase to facilitate the high-value utilization of keratin-containing wastes, have emerged as a crucial research focus in the field of keratin resource recovery.

As an important evolutionary tool, site-directed mutagenesis technology has been successfully applied to modify various enzymes, including enhancing enzymatic activity and stability. Based on the wild-type keratinase KT-ker derived from Streptomyces (the amino acid sequence is shown in SEQ ID NO: 1), the present disclosure constructed a mutant library thereof by error-prone PCR. Subsequently, expression elements such as the signal peptide, kasOp* promoter, and T1 terminator derived from Streptomyces coelicolor are operably linked to each keratinase mutant gene, and Streptomyces lividans TK24 is used as the host strain for expressing the fusion gene. By evaluating the keratin substrate degradation capacity of the fermentation supernatants from recombinant Streptomyces strains, a keratinase mutant designated KT1113 (V99A/E116N/I117G/Y160S) with significantly enhanced enzymatic activity was identified, representing a 28% increase in activity relative to the wild-type KT-ker.

Mutant KT1113 has amino acid substitutions at four positions relative to the wild-type keratinase KT-ker, corresponding to positions 99, 116, 117, and 160: the amino acid at position 99 is substituted from valine (V) to alanine (A) (V99A), the amino acid at position 116 is substituted from glutamic acid (E) to asparagine (N) (E116N), the amino acid at position 117 is substituted from isoleucine (I) to glycine (G) (1117G), and the amino acid at position 160 is substituted from tyrosine (Y) to serine(S) (Y160S).

In view of the above, an embodiment of the present disclosure provides a keratinase mutant, the amino acid sequence of which is as follows:

ETADATRASVAELARVSDAVLDADVPGTAWYTDAESGKLVVTADATVSAAELAQ LKKAAGDKAGAVEIKRTPGTFNKLIAGGEAIYAAGGGRSSLGFNARSSSGATYALTAGHC TNGASTWYTNSGQTSLAGTRAGTSFPGNDYGLIRHSNASAADGRVSLYNGSYRDITGAG NAYVGQTVQRSGSTTGLHSGRVTGLNATVNYGGGDIVSGLIQTNVCAEPGDSGGALFAG STALGLTSGGSGNCRTGGTTFFQPVTEALSAYGVSII (SEQ ID NO: 2), wherein the substitution sites are underlined.

The keratinase mutant provided by the present disclosure can effectively degrade keratin substrates and exhibits excellent catalytic degradation activity toward keratin. When expressed in a Streptomyces expression system, the fermentation supernatant of the recombinant strain demonstrates an enzymatic activity of 2127.58 U/mL, whereas the fermentation supernatant of the wild-type strain under identical culture system and conditions exhibits an enzymatic activity of 1662.17 U/mL. Compared with the wild-type protein, the enzymatic catalytic activity of the mutant is increased by 28%. Furthermore, the keratinase mutant of the present disclosure can use keratin-containing wastes such as feathers as direct substrates. When combined with the reducing agent DTT and using the fermentation supernatant of the recombinant strain as the crude enzyme solution, it can substantially decompose an entire feather within 24 hours, significantly improving the degradation efficiency of keratin-containing wastes such as feathers. Unlike traditional engineered strains fermented with carbohydrate-based carbon sources and organic nitrogen sources as medium components, this approach can significantly reduce the production cost of keratinase and the processing cost of keratin-containing wastes, making it more suitable for industrial applications. It has broad market prospects and industrial applicability in facilitating the degradation of feather wastes, as well as the conversion, recovery, and reuse of keratin resources.

In another embodiment of the present disclosure, there is provided a nucleic acid molecule containing a gene sequence encoding aforementioned keratinase mutant (V99A/E116N/I117G/Y160S).

The advantages of the nucleic acid molecule over the prior art are the same as those of the aforementioned keratinase mutant, and therefore are not reiterated herein.

The nucleic acid molecule includes DNA molecules (e.g., genomic DNA or cDNA) and/or RNA molecules (e.g., mRNA), and the molecule may be single-stranded or double-stranded.

The sequence of the nucleic acid molecule can be derived from the amino acid (AA) sequence of the keratinase mutant by conventional means, such as in accordance with the rules of codon encoding. The full-length sequence of the nucleic acid molecule or a fragment thereof can usually be obtained by PCR amplification, recombination, or artificial synthesis.

Illustratively, the gene (DNA) sequence encoding the aforementioned keratinase mutant is set forth in SEQ ID NO: 3.

It will be understood by those skilled in the art that, due to the degeneracy of the genetic code, gene sequences different from the exemplified sequence above can also encode the keratinase mutant of the present disclosure. Accordingly, the exemplified sequence should not be construed as limiting the protection scope of the present disclosure.

Optionally, the nucleic acid molecule further contains gene sequences encoding a signal peptide, a promoter, and/or a terminator. The keratinase mutant is located downstream of the signal peptide, the promoter is located upstream of the signal peptide, and the terminator is located downstream of the keratinase mutant. Specifically, when all the aforementioned expression elements are included, the gene linkage order from upstream to downstream is: promoter-signal peptide-keratinase mutant-terminator. The signal peptide is used to direct the secretory expression of the keratinase in the host strain, facilitating its separation and purification. The promoter is used to initiate the transcription process of the keratinase, and the terminator is used to terminate the transcription of the keratinase, so as to form a complete keratinase expression cassette. The signal peptide, promoter, and terminator are adaptively selected according to the types of the expression vector and host cell. Moreover, in the actual expression of the keratinase, each expression element may also be selected according to practical needs. For example, if secretory expression is not required or the selected vector already contains a signal peptide, there is no need to additionally add a signal peptide gene upstream of the keratinase mutant gene.

In yet another embodiment of the present disclosure, there is provided an expression vector comprising the aforementioned nucleic acid molecule.

Optionally, the expression vector includes a prokaryotic expression vector, a eukaryotic expression vector, or a viral expression vector (e.g., lentivirus, adenovirus). Correspondingly, the host cells transformed or transfected with the expression vector may be prokaryotic cells or eukaryotic cells, selected according to the type of the expression vector. For example, when a prokaryotic expression vector is used, prokaryotic cells are chosen as host cells, and common examples of prokaryotic cells include Escherichia coli, Bacillus spp., Corynebacterium spp., Zymomonas mobilis, and Streptomyces spp.; when a eukaryotic expression vector is used, eukaryotic cells are chosen as host cells, and common examples of eukaryotic cells include Saccharomyces cerevisiae, Saccharomyces pastorianus, and filamentous fungi.

Typical vectors include plasmids (e.g., pUC series, pET series, pWB series, pGEX series, pDXW series, pBR322, pEZ15A, pMA5, pPICZα, PIC9K, pSET152), viral vectors, bacteriophages (e.g., λgt4λB, λ Charon, λΔz1, and M13), cosmids, and minichromosomes. Plasmids are the most commonly used vectors; therefore, in the context of the present disclosure, plasmids and vectors are used interchangeably unless otherwise specified.

Streptomyces, as an important protein expression host, possesses advantages including mature industrial cultivation system, convenient genetic operation, high safety, strong metabolic activity, high protein secretion efficiency, abundant transcriptional signals, high heterologous protein yield, and no endotoxin production. These advantages endow it with broad application prospects and great development potential in the field of biotechnology. In an preferred embodiment of the present disclosure, a Streptomyces expression system is adopted. The expression vector used is the Streptomyces integrative vector pSET152, the host (or recipient) cell is Streptomyces lividans TK24, the signal peptide is the vsi signal peptide from Streptomyces coelicolor, the promoter is the kasOp* promoter from Streptomyces coelicolor, and the terminator is the Tl terminator from Streptomyces coelicolor. By operably linking the aforementioned expression elements with the keratinase mutant gene to construct an expression vector, and subsequently obtaining recombinant Streptomyces transformed with the expression vector, high-efficiency secretory expression of the keratinase can be achieved through conventional fermentation culture. The target enzyme accounts for more than 99% of the extracellular fermentation broth, thus enabling the fermentation supernatant of the recombinant strain to serve directly as a high-quality keratinase source for keratin hydrolysis. This approach enhances the production efficiency and reduces the production cost of keratinase, lowering the conversion and utilization costs of keratin-containing wastes such as feathers, and meeting the market demand for green and high-efficiency products.

Preferably, the gene (DNA) sequence of the aforementioned vsi signal peptide is shown in SEQ ID NO: 4, the gene (DNA) sequence of the kasOp* promoter is shown in SEQ ID NO: 5, and the gene (DNA) sequence of the T1 terminator is shown in SEQ ID NO: 6; the gene (DNA) sequence of the fusion gene formed by the aforementioned expression elements and the keratinase mutant is shown in SEQ ID NO: 7.

In a further embodiment of the present disclosure, there is provided a recombinant strain comprising the aforementioned nucleic acid molecule or the aforementioned expression vector.

Optionally, the parental strain of the recombinant strain is Streptomyces, specifically Streptomyces lividans TK24.

The introduction of the aforementioned nucleic acid molecule or expression vector into host cells via transformation or transfection can be carried out by conventional techniques well known in the art, including: CaCl₂)-mediated transformation, calcium phosphate co-precipitation method, electroporation, gene gun bombardment, microinjection, conjugative transfer, liposome-mediated transfection, liposome fusion, lipofection, and protoplast fusion, among others.

An embodiments of the present disclosure further provides a method for preparing the keratinase mutant, comprising:
(i) culturing the aforementioned recombinant strain;
(ii) centrifuging the fermentation broth to obtain a supernatant;
(iii) filtering the supernatant;
and (iv) collecting the filtrate to obtain the crude enzyme solution containing the keratinase mutant.

An embodiments of the present disclosure further provides the use of the aforementioned keratinase mutant in treating keratin-containing substances, as well as a related treatment method. The keratin-containing substances include, but are not limited to: feathers, hair, wool, and preferably avian feathers.

The method for treating keratin-containing substances with the keratinase mutant comprises:
(a) mixing the keratinase mutant or a crude enzyme solution thereof with the keratin-containing substances;
(b) adding a reducing agent, wherein the reducing agent may be dithiothreitol (DTT);
and (c) performing a catalytic reaction at 50 °C-60 °C.

Preferably, the concentration of the reducing agent in the reaction solution is usually 1-5 mM.

The present disclosure will be further described below in conjunction with specific examples. Unless otherwise specified, the experimental methods without explicitly specific conditions in the following examples are usually carried out under conventional conditions, such as those described in Molecular Cloning: A Laboratory Manual (4th Edition) published by Cold Spring Harbor Laboratory Press, or as recommended by the manufacturer.

In the examples described below, the main biological materials include:
(1) Strains: Escherichia coli DH5α is commercially available; Streptomyces lividans (S. lividans) TK24 was obtained from the Streptomyces Group of John Innes Centre (Norwich, NR4 7UH, England).
(2) Expression vector: The Streptomyces integrative vector pSET152 contains expression elements including int, phiC31 attP site, Factor Xa site, oriT-RP4, TraJ, aac (3) IV, and ori. Antibiotics used for screening were apramycin and nalidixic acid. Information on the pSET152 is available under GenBank Accession Number: AJ414670.1.
(3) Enzymes and kits for vector construction: T4 DNA Ligase (Cat. No.: C301-01), PCR reagent (2× KeyPo Master Mix (Dye Plus), Cat. No.: PK511-01), Plasmid Extraction Kit (Fastpure Plasmid Mini Kit-BOX 2, Cat. No.: DC201-01), Gel Purification and Recovery Kit (FastPure Gel DNA Extraction Mini Kit, Cat. No.: DC301-01), and One-Step Cloning Kit (ClonExpress Ultra One Step Cloning Kit V3, Cat. No.: C117-01), were purchased from Vazyme Biotech Co., Ltd. (Nanjing, China).
(4) Culture media:
(a) Escherichia coli medium (LB medium): 5 g/L yeast extract, 10 g/L tryptone, and 1 g/L NaCl, pH 7.0;
(b) Apramycin-resistant LB medium: LB medium supplemented with 50 μg/mL apramycin, pH 7.0;
(c) Kanamycin-resistant LB medium: LB medium supplemented with 50 μg/mL kanamycin, pH 7.0;
(d) Conjugative transfer medium (Modified-ISP4 medium): 10 g/L soluble starch, 1.0 g/L K₂HPO₄, 1.0 g/L NaCl, 2.0 g/L (NH₄)₂SO₄, 2.0 g/L CaCO₃, 0.5 g/L yeast extract, 1 g/L tryptone, 1 mL/L inorganic salt solution, and 20 g/L agar. The inorganic salt solution contains: 1 g/L FeSO₄, 1 g/L MnCl₂, and 1 g/L ZnSO₄, pH 7.2-7.4. Before use, MgCl₂ is added to the conjugative transfer medium to a final concentration of 30 mM;
(e) Protein secretion expression medium (TSB medium): 30 g/L tryptic soy broth, pH 7.2-7.4

### 1. Screening of the Keratinase Mutants

Based on the wild-type keratinase KT-ker, a keratinase mutant library was constructed by amplifying its encoding gene via error-prone PCR. Subsequently, the vsi signal peptide (amino acid sequence: MRRTLKAVGAAAAAATCVLAATAGTAQA), the strong promoter kasOp*, and the terminator T1, all derived from Streptomyces coelicolor, were operably linked to each keratinase mutant gene. The resulting fusion gene was then recombined with the integrative expression vector pSET152 to construct a recombinant expression vector capable of the secretory expression of the keratinase gene. This recombinant expression vector was introduced into Streptomyces lividans TK24 via the E. coli-Streptomyces conjugative transfer method, thereby achieving high-efficiency secretory expression. Thereafter, the keratinase activity in the fermentation supernatant of the recombinant strain was determined, and the combined keratinase mutant KT1113 with enhanced enzymatic activity was screened out. Compared with the wild-type keratinase KT-ker, KT1113 comprises the following amino acid substitutions: V99A/E116N/I117G/Y160S.

The amino acid sequence of the wild-type keratinase KT-ker is as follows:

ETADATRASVAELARVSDAVLDADVPGTAWYTDAESGKLVVTADATVSAAELAQ LKKAAGDKAGAVEIKRTPGTFNKLIAGGEAIYAAGGGRSSLGFNVRSSSGATYALTAGHC TEIASTWYTNSGQTSLAGTRAGTSFPGNDYGLIRHSNASAADGRVYLYNGSYRDITGAG NAYVGQTVQRSGSTTGLHSGRVTGLNATVNYGGGDIVSGLIQTNVCAEPGDSGGALFAG STALGLTSGGSGNCRTGGTTFFQPVTEALSAYGVSII (SEQ ID NO: 1). The amino acid residues to be mutated are indicated in italics.

The amino acid sequence of the keratinase mutant KT1113 is as follows:

ETADATRASVAELARVSDAVLDADVPGTAWYTDAESGKLVVTADATVSAAELAQ LKKAAGDKAGAVEIKRTPGTFNKLIAGGEAIYAAGGGRSSLGFNARSSSGATYALTAGHC TNGASTWYTNSGQTSLAGTRAGTSFPGNDYGLIRHSNASAADGRVSLYNGSYRDITGAG NAYVGQTVQRSGSTTGLHSGRVTGLNATVNYGGGDIVSGLIQTNVCAEPGDSGGALFAG STALGLTSGGSGNCRTGGTTFFQPVTEALSAYGVSII (SEQ ID NO: 2). The substituted amino acid residues are indicated by underlining.

The gene sequences used for constructing the expression vector ot keratinase are listed in Table 1.

TABLE 1
Genes and Primer Sequences Used in the Examples

| Sequence | |
|---|---|
| name | DNA sequence information |
| KT1113 | GAGACCGCCGACGCCACCCGCGCCTCCGTCGCCGAGCTGGCCCGCGTCTC |
| mutant | CGACGCCGTCCTGGACGCCGACGTCCCGGGCACCGCCTGGTACACCGACG |
| | CCGAGTCCGGCAAGCTGGTCGTCACCGCCGACGCCACCGTCTCCGCCGCC |
| | GAGCTGGCCCAGCTGAAGAAGGCCGCCGGCGACAAGGCCGGCGCCGTCG |
| | AGATCAAGCGCACCCCGGGCACCTTCAACAAGCTGATCGCCGGCGGCGAG |
| | GCCATCTACGCCGCCGGCGGCGGCCGCTCCTCCCTGGGCTTCAACGCGCGC |
| | TCCTCCTCCGGCGCCACCTACGCCCTGACCGCCGGCCACTGCACCAACGGC |
| | GCCTCCACCTGGTACACCAACTCCGGCCAGACCTCCCTGGCCGGCACCCGC |
| | GCCGGCACCTCCTTCCCGGGCAACGACTACGGCCTGATCCGCCACTCCAAC |
| | GCCTCCGCCGCCGACGGCCGCGTCTCGCTGTACAACGGCTCCTACCGCGAC |
| | ATCACCGGCGCCGGCAACGCCTACGTCGGCCAGACCGTCCAGCGCTCCGG |
| | CTCCACCACCGGCCTGCACTCCGGCCGCGTCACCGGCCTGAACGCCACCGT |
| | CAACTACGGCGGCGGCGACATCGTCTCCGGCCTGATCCAGACCAACGTCT |
| | GCGCCGAGCCGGGCGACTCCGGCGGCGCCCTGTTCGCCGGCTCCACCGCC |
| | CTGGGCCTGACCTCCGGCGGCTCCGGCAACTGCCGCACCGGCGGCACCAC |
| | CTTCTTCCAGCCGGTCACCGAGGCCCTGTCCGCCTACGGCGTCTCCATCAT |
| | CTGA (SEQ ID NO: 3) |
| vsi signal | ATGCGTCGCACCCTCAAGGCCGTGGGAGCAGCCGCGGCGGCGGCCACCTG |
| peptide | CGTCCTCGCCGCGACGGCAGGCACCGCGCAGGCC (SEQ ID NO: 4) |
| kasOp* | TGTTCACATTCGAACGGTCTCTGCTTTGACAACATGCTGTGCGGTGTTGTA |
| promoter | AAGTCGTGGCCAGGAGAATACGACAGCGTGCAGGACTGGGGGAGTT (SEQ |
| | ID NO: 5) |
| T1 | AGCAGTGCTGAAGGCCGTCAAGGGGCGTTCGTGGTGGGAAGA |
| terminator | (SEQ ID NO: 6) |
| KT1113 | TGTTCACATTCGAACGGTCTCTGCTTTGACAACATGCTGTGCGGTGTTGTAA |
| expression | AGTCGTGGCCAGGAGAATACGACAGCGTGCAGGACTGGGGGAGTTATGCG |
| cassette | TCGCACCCTCAAGGCCGTGGGAGCAGCCGCGGCGGCGGCCACCTGCGTCC |
| | TCGCCGCGACGGCAGGCACCGCGCAGGCCGAGACCGCCGACGCCACCCGC |
| | GCCTCCGTCGCCGAGCTGGCCCGCGTCTCCGACGCCGTCCTGGACGCCGAC |
| | GTCCCGGGCACCGCCTGGTACACCGACGCCGAGTCCGGCAAGCTGGTCGT |
| | CACCGCCGACGCCACCGTCTCCGCCGCCGAGCTGGCCCAGCTGAAGAAGG |
| | CCGCCGGCGACAAGGCCGGCGCCGTCGAGATCAAGCGCACCCCGGGCACC |
| | TTCAACAAGCTGATCGCCGGCGGCGAGGCCATCTACGCCGCCGGCGGCGG |
| | CCGCTCCTCCCTGGGCTTCAACGCGCGCTCCTCCTCCGGCGCCACCTACGC |
| | CCTGACCGCCGGCCACTGCACCAACGGCGCCTCCACCTGGTACACCAACTC |
| | CGGCCAGACCTCCCTGGCCGGCACCCGCGCCGGCACCTCCTTCCCGGGCA |
| | ACGACTACGGCCTGATCCGCCACTCCAACGCCTCCGCCGCCGACGGCCGCG |
| | TCTCGCTGTACAACGGCTCCTACCGCGACATCACCGGCGCCGGCAACGCCT |
| | ACGTCGGCCAGACCGTCCAGCGCTCCGGCTCCACCACCGGCCTGCACTCC |
| | GGCCGCGTCACCGGCCTGAACGCCACCGTCAACTACGGCGGCGGCGACAT |
| | CGTCTCCGGCCTGATCCAGACCAACGTCTGCGCCGAGCCGGGCGACTCCG |
| | GCGGCGCCCTGTTCGCCGGCTCCACCGCCCTGGGCCTGACCTCCGGCGGCT |
| | CCGGCAACTGCCGCACCGGCGGCACCACCTTCTTCCAGCCGGTCACCGAG |
| | GCCCTGTCCGCCTACGGCGTCTCCATCATCTGAAGCAGTGCTGAAGGCCGT |
| | CAAGGGGCGTTCGTGGTGGGAAGA (SEQ ID NO: 7) |
| Vector-F | CCATCATCTGAAGCAGTGCTGAAGGCCGTCAAG (SEQ ID NO: 8) |
| Vector-R | GTGCGACGCATAACTCCCCCAGTCCTGCACGCTG (SEQ ID NO: 9) |
| KT-keratin- | GGGGGAGTTATGCGTCGCACCCTCAAGGCCG (SEQ ID NO: 10) |
| F | |
| KT-keratin- | CAGCACTGCTTCAGATGATGGAGACGCCGTAG (SEQ ID NO: 11) |
| R | |
| error-prone- | CTGGGGGAGTTATGCGTCGCAC (SEQ ID NO: 12) |
| F | |
| error-prone- | CAGCACTGCTTCAGATGATGG (SEQ ID NO: 13) |
| R | |

### 1.1 Construction of Wild-Type Keratinase Expression Vector

Construction of the base vector pSET152-kasOp-T1: The fusion fragment kasOp*-T1, comprising the kasOp* promoter (sequence shown in SEQ ID NO: 5) and the T1 terminator (sequence shown in SEQ ID NO: 6), was synthesized by Tsingke Biotechnology Co., Ltd. A restriction enzyme cleavage site for BamHI was added upstream of the kasOp* promoter, and a restriction enzyme cleavage site for EcoRV was added downstream of the T1 terminator. The kasOp*-T1 fragment and the pSET152 vector were double-digested separately with restriction enzymes BamHI and EcoRV. After purification and recovery, the kasOp*-T1 fragment was ligated to the pSET152 expression vector, and the ligation product was transformed into Escherichia coli DH5a. Following sequence verification to confirm correctness, the base expression vector pSET152-kasOp*-T1 was obtained.

The gene encoding the keratinase (KT-ker) derived from Streptomyces was optimized according to Streptomyces codon preference, and the secretory signal peptide vsi (sequence shown in SEQ ID NO: 4) was added to its N-terminus. The optimized nucleotide sequence was synthesized by Tsingke Biotechnology Co., Ltd. The keratinase gene with the vsi signal peptide at its N-terminus was designated SSvsi-KT-ker.

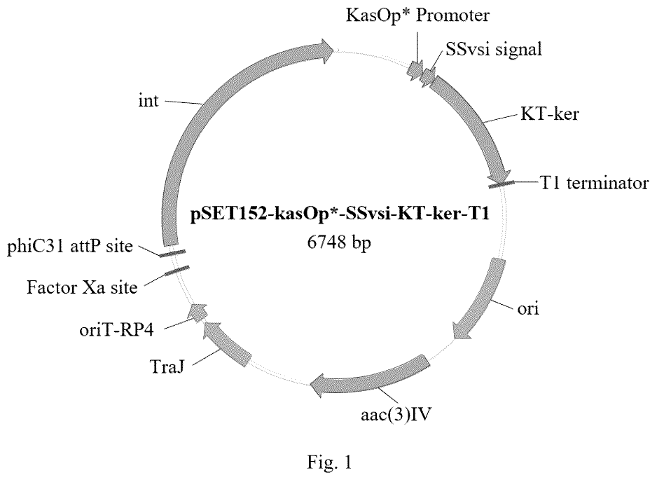

Using high-fidelity DNA polymerase, the linearized pSET152-kasOp*-T1 vector was amplified by polymerase chain reaction (PCR) with the primer pair Vector-F (sequence shown in SEQ ID NO: 8) and Vector-R (sequence shown in SEQ ID NO: 9). Subsequently, the SSvsi-KT-ker gene was amplified with the primer pair KT-keratin-F (sequence shown in SEQ ID NO: 10) and KT-keratin-R (sequence shown in SEQ ID NO: 11). The PCR products were purified using a gel purification kit, and the linearized pSET152-kasOp*-T1 and SSvsi-KT-ker gene fragment were ligated via a one-step cloning kit. The ligation product was then transformed into Escherichia coli DH5a, and the transformed strains were cultured on LB medium supplemented with apramycin for selection. Positive clones were screened and confirmed by sequencing, resulting in the recombinant expression vector pSET152-kasOp*-SSvsi-KT-ker-T1 that harbored the correct keratinase KT-ker gene sequence. Its vector map is shown in FIG. 1, where KT-ker denotes the wild-type keratinase gene, SSvsi signal denotes the vsi signal peptide gene, kasOp* promoter denotes the kasOp* promoter gene, and T1 terminator denotes the T1 terminator gene.

### 1.2 Construction of Keratinase Mutant Library

Random mutations were introduced via error-prone PCR to perform protein engineering on the keratinase and screen for mutants. Primers error-prone-F (sequence shown in SEQ ID NO: 12) and error-prone-R (sequence shown in SEQ ID NO: 13) were designed for random PCR mutagenesis. Using the wild-type keratinase KT-ker gene as the template, PCR amplification was conducted with the aforementioned error-prone PCR primers using a random mutagenesis PCR kit (GeneMorph II Random Mutagenesis Kit, purchased from Biomars), which can generate approximately equal probabilities of A, T, G, and C mutations during amplification. After amplification, the amplified product was recovered and ligated to the linearized pSET152-kasOp*-T1 vector. The ligation products were transformed into Escherichia coli DH5a, and transformants spread on apramycin-resistant LB plates, followed by inverted incubation at 37 °C.

### 1.3 Construction of Recombinant Strains for the Keratinase Mutant Library

The expression vectors of the keratinase mutant library were introduced into the host Streptomyces cells via the conjugative transfer method. The specific procedures are as follows:
(1) Single colonies of transformants grown on apramycin-resistant LB plates were picked into a 96-well plate, with 50 μL of apramycin-resistant LB medium added to each well. The plate was incubated at 37 °C with shaking at 220 rpm for approximately 6 hours. The bacterial cells in the plate were mixed and transferred to 50 mL of apramycin-resistant LB medium for scale-up cultivation, and further incubated at 37 °C with shaking at 220 rpm for about 3 hours until the OD₆₀₀ reached 0.5-0.6. The cells were harvested by centrifugation at 5000 rpm for 4 minutes, washed once with LB medium, and finally resuspended in 2 mL of LB medium to obtain the donor E. coli DH5α recombinant bacterial suspension.
(2) The E. coli-Streptomyces triparental conjugative transfer helper strain ET12567/pUB307 was inoculated. A 500 μL aliquot of the overnight culture was transferred to 50 mL of LB medium supplemented with kanamycin and chloramphenicol, and incubated at 37 °C with shaking at 220 rpm for approximately 3 hours until the OD₆₀₀ reached 0.5-0.6. The cells were harvested by centrifugation at 5000 rpm for 4 minutes, washed once with LB medium, and finally resuspended in 2 mL of LB medium to obtain the helper E. coli ET12567/PUB307 suspension.
(3) 2 mL aliquot of the spore suspension of Streptomyces lividans TK24 was centrifuged at 10000 rpm for 2 minutes. The glycerol was discarded, and the spores were washed once with TSB medium and resuspended in 10 mL of TSB medium. The suspension was heat-shocked in a water bath at 50 °C for 10 minutes, further recovered with shaking at 30 °C for 3 hours. Subsequently, the spores were washed once with TSB medium and resuspended in 1 mL of TSB medium to obtain the recipient S. lividans TK24 suspension.
(4) 200 μL of the donor E. coli DH5α recombinant bacterial suspension, 200 μL of the helper E. coli ET12567/pUB307 suspension, and 100 μL of the recipient S. lividans TK24 suspension were mixed thoroughly. The mixture was spread on conjugative transfer medium plates (supplemented with MgCl₂ to a final concentration of 30 mM before pouring) and incubated at 28 °C for 16-20 hours. Thereafter, the plates were overlaid with a solution containing apramycin (stock solution: 50 mg/mL) and nalidixic acid (stock solution: 25 mg/mL). Specifically, 25 μL of apramycin and 50 μL of nalidixic acid were added to 1 mL of sterile water, mixed well, and then overlaid onto the entire plate, resulting in a final concentration of 50 μg/mL apramycin and 50 μg/mL nalidixic acid in the plates. The plates were air-dried in a clean bench for approximately 1 hour until completely dry. Afterwards, they were cultured at 28 °C for 5-7 days, and the resulting conjugants were picked and streaked on antibiotic selection plates containing apramycin and nalidixic acid for single-colony isolation.

### 1.4 Screening of the Keratinase Mutants

Single colonies from the aforementioned selection plates were picked into a 96-well plate, with 200 μL of TSB medium added to each well. The plate was incubated at 28 °C with shaking at 220 rpm for approximately 96 hours. The supernatant of the fermentation broth was then collected by centrifugation for keratinase activity assay.

The keratinase activity assay was performed with reference to the national recommended standard “Spectrophotometric Method for the Determination of Acidic and Neutral Protease Activity in Feed Additives (Standard No.: GB/T 28715-2012)” and the Ministry of Agriculture and Rural Affairs recommended standard “Spectrophotometric Method for the Determination of Keratinase Activity (Standard No.: NY/T 4362-2023)”. The principle of the assay is as follows: Keratinase hydrolyzes keratin under specific temperature and pH conditions (unless otherwise specified, the temperature is 60 °C and pH is 10 in the present disclosure), producing amino acids containing phenolic groups. These amino acids are reduced by the Folin-phenol reagent to form tungsten blue. The absorbance of the solution was measured at 680 nm using an ultraviolet spectrophotometer. Since the enzyme activity is proportional to the absorbance, the specific activity of keratinase can be calculated. The assay method is as follows:

Blank group: 20 μL of fermentation supernatant was transferred to a new 96-well plate, incubated at 60 °C for 2 minutes, and then 40 μL of trichloroacetic acid (TCA) was added. After the mixture reacted at 60 °C for 10 minutes, 20 μL of a solution containing 10 g/L feather fragments was added. Subsequently, the plate was taken out and allowed to stand for 10 min, followed by centrifugation at 12000 r/min for 2 min. 50 μL of the supernatant was collected, mixed with 50 μL of Na₂CO₃ solution, and then 10 μL of Folin-Ciocalteu reagent was added. After color development at 60 °C for 20 minutes, the absorbance of the solution was measured at 680 nm using an ultraviolet spectrophotometer. Trichloroacetic acid is used to inactivate the enzyme, and the blank group is used to subtract the background value.

Sample group: 20 μL of fermentation supernatant was transferred to a new 96-well plate, incubated at 60 °C for 2 minutes, and then 20 μL of a solution containing 10 g/L feather fragments was added. After reacting at 60 °C for 10 minutes, 40 μL of trichloroacetic acid (TCA) was added to terminate the reaction. Subsequently, the plate was taken out and allowed to stand for 10 min, followed by centrifugation at 12000 r/min for 2 min. 50 μL of the supernatant was collected, mixed with 50 μL of Na₂CO₃ solution, and then 10 μL of Folin-Ciocalteu reagent was added. After color development at 60 °C for 20 minutes, the absorbance of the solution was measured at 680 nm using an ultraviolet spectrophotometer.

The keratinase activity secreted in the supernatants of recombinant strains harboring different mutants was determined by comparing the absorbance (OD) values of different fermentation supernatant samples. For recombinant Streptomyces strains with significantly increased keratinase activity, genomic DNA was extracted for PCR amplification to obtain the mutated gene fragments. The fragments were sequenced, and the mutant KT1113 (V99A/E116N/I117G/Y160S) was screened out, which significantly enhances keratinase activity without affecting its inherent enzymatic properties.

### 2. Enzymatic Activity Analysis of Wild-Type and Mutant Keratinase

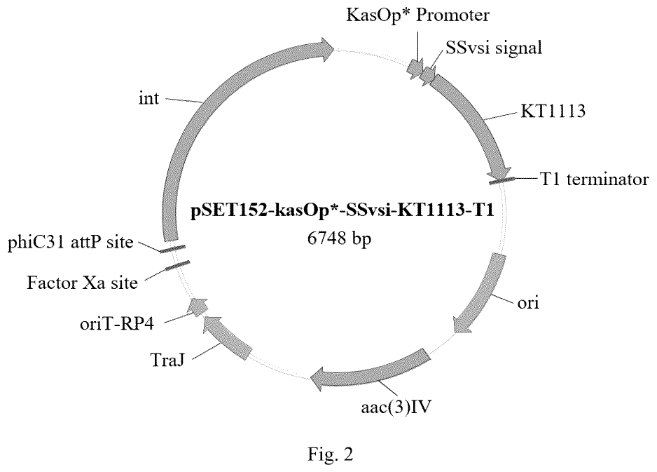

The mutated gene fragments obtained from the aforementioned amplification were ligated to the linearized pSET152-kasOp*-T1 vector and transformed into Escherichia coli DH5a. To ensure accuracy, several clones were sequenced. Plasmids were purified from E. coli clones that yielded correct sequencing results, thereby obtaining the mutant expression vector pSET152-kasOp-SSvsi-KT1113-T1. Its vector map is shown in FIG. 2, where KT1113 denotes the keratinase mutant gene, SSvsi signal denotes the vsi signal peptide gene, kasOp* promoter denotes the kasOp* promoter gene, and T1 terminator denotes the T1 terminator gene.

The expression vector pSET152-kasOp*-SSvsi-KT1113-T1 was transferred into the recipient Streptomyces lividans TK24 cells via conjugative transfer to obtain the mutant recombinant strain, designated as TK24::pSET152-kasOp*-SSvsi-KT1113-T1. Single colonies of the mutant recombinant strain were picked into a 96-well plate, each well was supplemented with 200 μL of TSB medium. The plate was incubated at 28 °C with shaking at 220 rpm for 96 hours. The fermentation broth was centrifuged at 12000 rpm for 15 minutes, and the supernatant was filtered through a 0.22 μm membrane using a positive-pressure filter to obtain the keratinase fermentation supernatant, which was used as the crude enzyme solution for determining the mutant enzyme activity. Streptomyces lividans TK24 transformed with pSET152-kasOp*-SSvsi-KT-ker-T1 (TK24::pSET152-kasOp*-SSvsi-KT-ker-T1) was used as the control strain.

The results of enzyme activity assays for the crude enzyme solutions of the mutant and wild-type keratinases are presented in Table 2. As shown, the enzyme activity of the quadruple-mutant keratinase KT1113 is 2127.58 U/mL, whereas that of the wild-type keratinase is 1662.17 U/mL. The enzyme activity of KT1113 is 28% higher than that of the wild-type KT-ker, confirming that the keratinase mutant KT1113 exhibited significantly improved enzymatic activity after protein engineering modification.

TABLE 2
Enzymatic Activities of Crude Enzyme Solutions of Mutant
and Wild-Type Keratinase (Test Conditions: 60 °C, pH 10)

| Keratinase | Genotype/Mutation Site | Enzymatic Activity (U/mL) |
|---|---|---|
| KT-ker | Wild-type | 1662.17 |
| KT1113 | V99A/E116N/I117G/Y160S | 2127.58 |

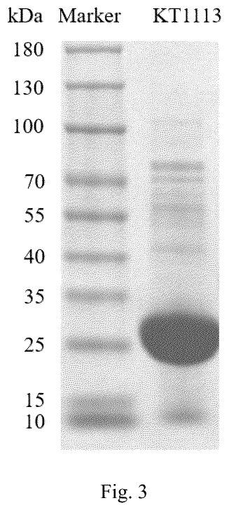

In addition, sodium dodecyl sulfate-polyacrylamide gel electrophoresis (SDS-PAGE) was performed on the crude mutant enzyme solution to verify the molecular weight and expression level of the secreted keratinase mutant KT1113, and the result is shown in FIG. 3. A distinct keratinase band was observed at approximately 25 kDa in the fermentation supernatant, which is consistent with its theoretical molecular weight of 26.7 kDa. Meanwhile, the keratinase concentration in the supernatant is high, reaching 1.45 g/L, with a purity exceeding 99%.

### 3. Establishment and Effect Evaluation of the Feather Degradation Process Using the Keratinase Mutant

10 μL of the spore suspension of TK24::pSET152-kasOp*-SSvsi-KT1113-T1 was inoculated into a PA bottle containing 5 mL of TSB medium supplemented with 25 μg/mL apramycin, and cultured at 28 °C for 2 days to prepare a seed culture. 2 mL of the seed culture was transferred to 50 mL of TSB medium and cultured at 28 °C for 4 days. The culture was centrifuged, and the supernatant was collected as the fermentation supernatant of the recombinant keratinase. The fermentation supernatant was subsequently filtered through a 0.22 μm membrane using a positive-pressure filter to obtain the crude keratinase solution.

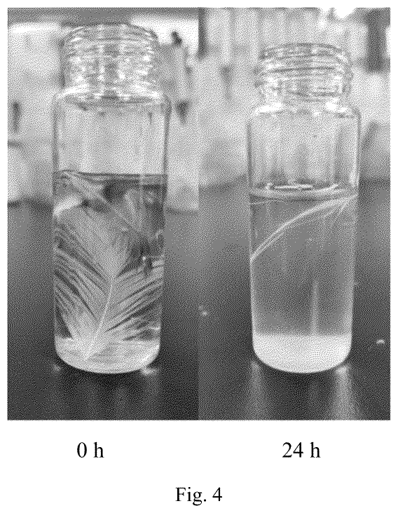

A reducing agent (dithiothreitol, DTT) was added to the crude keratinase solution to a final concentration of 2 mM, followed by the addition of an intact feather. The feather degradation was tested by enzymatic hydrolysis at 50 °C. The results are shown in FIG. 4, where the left panel is a photograph of the feather at 0 hours after being placed in the KT1113 keratinase solution, and the right panel is a photograph of the feather after 24 hours of incubation with the KT1113 keratinase solution at 50 °C. As shown, the keratinase mutant exhibits excellent feather degradation efficiency, with the majority of the feather being degraded within 24 hours. This indicates that the recombinant keratinase KT1113, in combination with the reducing agent DTT, can efficiently degrade feathers.

Feathers contain approximately 80% crude protein, making them a potential high-quality protein resource. The products of feather waste degraded by keratinase are rich in amino acids (such as cystine, glycine, proline) and polypeptide nutrients, which have been proven to partially replace relatively expensive protein feeds like fish meal and soybean meal. Furthermore, this process can alleviate environmental pollution caused by feather waste. Therefore, the development of high-activity keratinase holds significant practical importance and research value. The quadruple-mutant keratinase KT1113 derived from Streptomyces provided by the present disclosure can directly degrade feathers through a mild reaction in the presence of DTT. Compared with traditional keratinase-producing engineered strains that rely on fermentation with carbohydrate-based carbon sources and organic nitrogen sources, this approach can significantly reduce the conversion cost of keratin, addresses the challenge of efficient and low-cost hydrolysis of feathers, and offers advantages such as high degradation efficiency and environmental friendliness.

The foregoing descriptions are merely preferred embodiments of the present disclosure, and are not intended to limit it. Any modifications, equivalent replacements and improvements made within the spirit and principles of the present disclosure shall fall within the protection scope of the present disclosure.

What is claimed is:

1. A keratinase mutant, wherein the amino acid sequence of the keratinase mutant is set forth in SEQ ID NO: 2.

2. A nucleic acid molecule, containing a gene sequence encoding the keratinase mutant according to claim 1.

3. The nucleic acid molecule according to claim 2, wherein the gene sequence encoding the keratinase mutant is set forth in SEQ ID NO: 3.

4. The nucleic acid molecule according to claim 2, further containing gene sequences encoding a signal peptide, a promoter, and a terminator;
wherein the keratinase mutant is located downstream of the signal peptide, the promoter is located upstream of the signal peptide, and the terminator is located downstream of the keratinase mutant.

5. The nucleic acid molecule according to claim 4, wherein: the signal peptide is a vsi signal peptide with the gene sequence set forth in SEQ ID NO: 4; the promoter is a kasOp* promoter with the gene sequence set forth in SEQ ID NO: 5; and the terminator is a T1 terminator with the gene sequence set forth in SEQ ID NO: 6.

6. An expression vector or a recombinant strain, wherein the recombinant strain carries the expression vector, the expression vector comprises the nucleic acid molecule according to claim 3.

7. The expression vector or the recombinant strain according to claim 6, wherein the nucleic acid molecule further containing gene sequences encoding a signal peptide, a promoter, and a terminator;
wherein the keratinase mutant is located downstream of the signal peptide, the promoter is located upstream of the signal peptide, and the terminator is located downstream of the keratinase mutant.

8. The expression vector or the recombinant strain according to claim 7, wherein: the signal peptide is a vsi signal peptide with the gene sequence set forth in SEQ ID NO: 4; the promoter is a kasOp* promoter with the gene sequence set forth in SEQ ID NO: 5; and the terminator is a T1 terminator with the gene sequence set forth in SEQ ID NO: 6.

9. The expression vector or the recombinant strain according to claim 6, wherein the expression vector is a Streptomyces integrative vector pSET152, and the recombinant strain is Streptomyces lividans TK24.

10. Use of the keratinase mutant according to claim 1 in the treatment of keratin-containing substances.

11. The use according to claim 10, wherein the keratin-containing substances are selected from the group consisting of feathers, hair, or wool.

12. The use according to claim 11, wherein the keratin-containing substances are avian feathers.

13. The use according to claim 10, a method for treating the keratin-containing substances with the keratinase mutant comprising:
(a) mixing the keratinase mutant or a crude enzyme solution thereof with the keratin-containing substances;
(b) adding a reducing agent, wherein the reducing agent is dithiothreitol;
and (c) performing a catalytic reaction at a temperature ranging from 50 °C to 60 °C.
